(19) United States
(12) Patent Application Publication
(10) Pub. No.: US 2012/0315480 A1
(43) Pub. Date: Dec. 13, 2012

(54) COPPER NANOPARTICLES WITH MAGNETIC PROPERTIES

(75) Inventors: José Javier Saiz Garitaonandia, Leioa-Vizcaya (ES); Eider Goikolea Núñez, Leioa-Vizcaya (ES); Teófilo Rojo Aparicio, Leioa-Vizcaya (ES); Maite Insausti Peña, Leioa-Vizcaya (ES); Fernando Plazaola Muguruza, Leioa-Vizcaya (ES)

(73) Assignee: UNIVERSIDAD DEL PAIS VASCO, Leioa-Vizcaya (ES)

(21) Appl. No.: 13/387,970

(22) PCT Filed: Jul. 31, 2009
(86) PCT No.: PCT/ES2009/070326

§ 371 Date: Jul. 9, 2012

(51) Int. Cl. C07F 1/08; B22F 1/00; B01J 31/02; B82Y 15/00; B82Y 5/00; B82Y 30/00; B82Y 40/00

(52) U.S. Cl. 428/402; 556/110; 556/113; 502/165; 977/773; 977/896; 977/915; 977/902; 977/908; 977/927; 977/920; 977/842

(57) ABSTRACT

The present invention relates to thiol- or an amine-associated ferromagnetic or superparamagnetic copper nanoparticles with an average diameter less than 30 nm, to the method for obtaining them and their applications in biomedicine and other fields.

### FIELD OF THE INVENTION

The invention relates to the formation of copper nanoparticles having superparamagnetic or ferromagnetic properties. The invention also relates to a method for preparing same and the uses thereof.

### STATE OF THE ART

Surface functionalization of nanoparticles has aroused great interest since it allows, in addition to modifying their properties, the use thereof in a biological medium, broadening their applicability to biomedical environment. It has been observed that properties such as optical response, magnetism or reactivity differ significantly from those observed in massive state samples and that the changes in said properties can be modulated by the chemical affinity of the functional ligand to the surface of the metal nanoparticle.

Two pioneering works appear in 2004 in which it is shown for the first time that gold nanoparticles coated by organic ligands can have intrinsic magnetic moments [Crespo, P.; Litrán, R.; Rojas, T. C.; Multigner, M.; de la Fuente, J. M.; Sánchez-López, J. C.; García, M. A.; Hernando, A.; Penadés, S.; Fernández, A. Phys. Rev. Lett., 2004, 93, 087204/1-087204/4; Yamamoto, Y.; Miura, T.; Suzuki, M.; Kawamura, N.; Miyagawa, H.; Nakamura, T.; Kobayashi, K.; Teranishi, T.; Hori, H. Phys. Rev. Lett., 2004, 93, 116801/1-116801/4; Hori, H.; Yamamoto, Y.; Iwamoto, T.; Miura, T.; Teranishi, T.; Miyake, M. Phys. Rev. B, 2004, 69, 174411/1-174411/5]. From that time there have been many research groups which have confirmed the change from diamagnetism to magnetism by means of their experimental contributions, magnetic always being understood as superparamagnetic behavior or permanent magnetism or ferromagnetism [Negishi, Y.; Tsunoyama, H.; Suzuki, M.; Kawamura, N.; Matsushita, M. M.; Maruyama, K.; Sugawara, T.; Yokoyama, T.; Tsukuda, T. J. Am. Chem. Soc., 2006, 128, 12034-12035; Suda, M.; Kameyama, N.; Suzuki, M.; Kawamura, N.; Einaga, Y. Angew. Chem. Int. Ed., 2008, 47, 160-163.]. Also see WO 2005/091704. A2).

The possibility of extending this magnetic behavior change to other metals establishing metal-sulfur interactions was subsequently observed. Among the systems where this interaction has been most profoundly studied include self assembled monolayers (SAMs) supported on metals. The most widely used metals in this group were gold and palladium [Love, J. C.; Estroff, L. A.; Kriebel, J. K.; Nuzzo, R. G.; Whitesides, G. M. Chem. Rev., 2005, 105, 1103-1169]. The fact that gold and copper can have comparable properties in qualitative terms has also been seen in the field of SAMs [Laibinis, P. E.; Whitesides, G. M.; Allara, D. L.; Tao, Y. T.; Parikh, A. N.; Nuzzo, R. G. J. Am. Chem. Soc., 1991, 113, 7152-7167. (b) Walczak, M. M.; Chung, C.; Stole, S. M.; Widrig, C. A.; Porter, M. D. J. Am. Chem. Soc., 1991, 113, 2370-2378]. Ferromagnetic behavior has even been observed in silver and copper nanoparticles [Garitaonandia, J. S.; Insausti, M.; Goikolea, E.; Suzuki, M.; Cashion, J. D.; Kawamura, N.; Ohsawa, H.; Gil de Muro, I.; Suzuki, K.; Plazaola, F.; Rojo, T. Nano Lett., 2008, 8, 661-667], which, however, did not provide the reproducibility level required for practical applications. Gold nanoparticles have also been prepared in which an amine has been used as a surfactant (Leff, D. V., Brandt, L.; Heath, J. R. Langmuir, 1996, 12, 4723-4730).

Magnetic nanoparticles are susceptible to being “manipulated” by an external magnetic field, enabling the transportation and/or immobilization of same or of bound biological entities. Furthermore, the magnetic nanoparticle capacity to generate an energy transfer would allow the application thereof in treating tumors by means of hyperthermia. They can also have application as contrast agents in systems for obtaining images by means of magnetic resonance (Magnetic Resonance Imaging). Another biomedical application of the magnetic nanoparticles can include biological sample analysis by means of optical and electron spectroscopy.

The magnetic nanoparticles would also have application in very diverse fields different from the biomedical field such as their use as catalysts for synthesizing carbon nanotubes and inorganic nanowires or as a base for ultrathin systems for magnetic data storage.

### BRIEF DESCRIPTION OF THE DRAWINGS

FIG. 1 shows the evolution of magnetization of the nanoparticles of the invention with respect to the magnetic field. The y-axis shows the magnetic field and the x-axis shows the magnetization of the nanoparticles of the invention.

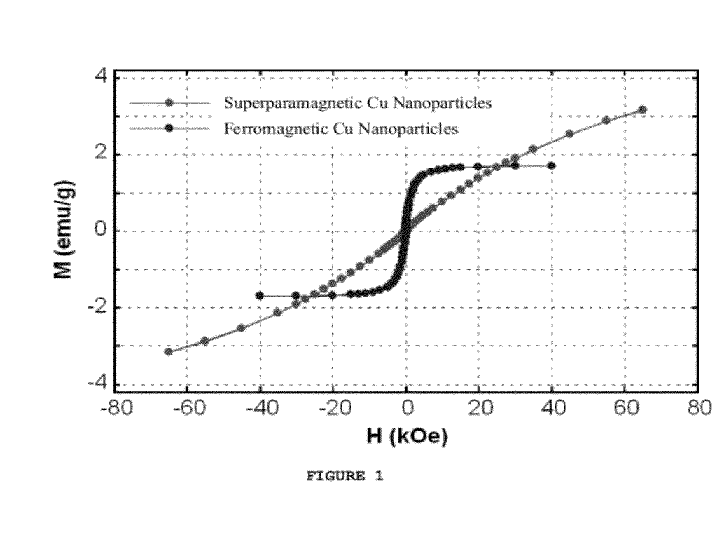

### BRIEF DESCRIPTION OF THE INVENTION

The inventors have prepared a family of copper-based nanoparticles with excellent magnetic properties. The nanoparticles of the invention have a permanent magnetic moment (ferromagnetic particles) or are superparamagnetic.

Therefore, a first aspect of the invention relates to a method for preparing a ferromagnetic nanoparticle which comprises
(i) providing a two-phase mixture of water and an organic solvent comprising a copper salt and a transfer agent;
(ii) adding a surfactant of formula RSH, where R is a hydrophobic group in the presence of a reducing agent; and
(iii) precipitating the nanoparticles formed
   said method is characterized in that the concentration of the copper salt in the aqueous phase is greater than 10⁻³ molar.

A second aspect is a ferromagnetic nanoparticle obtainable by said method.

A third aspect of the invention relates to a method for preparing a superparamagnetic nanoparticle which comprises
(i) providing a two-phase mixture of water and an organic solvent comprising a copper salt and a transfer agent;
(ii) adding a surfactant of formula RNH₂, where R is a hydrophobic group in the presence of a reducing agent; and
(iii) precipitating the nanoparticles formed.

A fourth aspect of the invention relates to a superparamagnetic nanoparticle with an average diameter less than 30 nm comprising copper associated with a surfactant of formula RNH₂, where R is a hydrophobic group, preferably, an alkyl, alkenyl, or alkynyl with 6 to 30 carbon atoms, more preferably an alkylamine of 6 to 30 carbon atoms.

For the purposes of the present application these nanoparticles are called “nanoparticles of the invention” and the methods for obtaining them are called “method of the invention”.

The nanoparticles of the invention have application in different fields, for example, the field of biomedicine. In a fifth aspect the invention relates to the nanoparticles of the invention for use as a medicament. Specifically, an additional aspect is the use of the nanoparticles of the invention for preparing a medicament or kit for (or the nanoparticles of the invention for) the transportation and/or immobilization of active ingredients in a biological medium, the controlled release of active ingredients, the treatment of tumors by means of hyperthermia, contrast agents in systems for obtaining images by means of magnetic resonance, or the analysis of biological samples by means of optical and electron spectroscopy.

Another additional aspect relates to the use of the nanoparticles of the invention as catalysts for synthesizing carbon nanotubes and inorganic nanowires, preparing biosensors and biochips, or for preparing ultrathin systems for magnetic data storage.

Copper is known for its tendency to oxidize. However, unlike what would be expected, the nanoparticles of the invention are stable and their oxidation is not observed. Preparing nanoparticles in a reproducible manner has also been achieved by means of the methods of the invention. These features, together with the fact that copper is economically viable, makes the nanoparticles of the invention an improvement with respect to known magnetic gold- and platinum-based nanoparticles or other copper-based nanoparticles.

Additional aspects of the invention are pharmaceutical compositions and kits comprising the nanoparticles of the invention.

### DETAILED DESCRIPTION

### Nanoparticles

According to a particular embodiment, the nanoparticles of the invention have between 10 and 90% organic material, particularly between 20 and 70%, more particularly between 25 and 60%.

The nanoparticles of the invention have an average diameter less than 30 nm, particularly between 1 and 20 nm, more particularly between 1 and 10 nm. Normally, the particle size obtained ranges between 2 and 5 nm.

Surfactants of formula RSH and RNH₂ associate with copper through the sulfur and nitrogen atoms, respectively, orienting their hydrophobic part outwards, thus forming the nanoparticles. Therefore, the hydrophobic chain must be large enough as to form the nanoparticles, preferably a hydrocarbon chain between 6 and 30 carbon atoms. Therefore, in a particular embodiment, R comprises between 6 and 20 carbon atoms. In another particular embodiment, it comprises between 8 and 15 carbon atoms, more preferably between 8 and 12 carbon atoms. According to a particular embodiment, R is linear. According to a particular embodiment, the compound of formula RNH₂ is 1-dodecylamine. According to a particular embodiment, the compound of formula RSH is 1-dodecanethiol.

The nanoparticles of the invention are magnetic nanoparticles. According to a preferred embodiment the saturation magnetization (Ms) is comprised between 0.01 and 3 emu/g_{Cu}, preferably between 0.1 and 3 emu/g_{Cu}.

For the purposes of the present invention hydrocarbon chain is understood as a linear or branched chain made up of carbon and hydrogen atoms which can contain unsaturations.

“Alkyl” refers to a radical of linear or branched hydrocarbon chain consisting of carbon and hydrogen atoms which does not contain unsaturation, and which is bound to the rest of the molecule by means of a single bond, for example, methyl, ethyl, n-propyl, i-propyl, n-butyl, t-butyl, n-pentyl, etc.

“Alkenyl” refers to a radical of linear or branched hydrocarbon chain consisting of carbon and hydrogen atoms, containing 1, 2 or 3 conjugated or non-conjugated carbon-carbon double bonds, such as —CH═CH₂, —CH₂CH═CH₂, —CH═CH—CH₂, —C(CH₃)═CH₂, —CH═CH—CH═CH₂, and the like.

“Alkynyl” refers to a radical of linear or branched hydrocarbon chain consisting of carbon and hydrogen atoms containing 1, 2 or 3 conjugated or non-conjugated carbon-carbon triple bonds, such as —CCH, —CH₂CCH, —CCCH₃, —CH₂CCCH₃, and the like

### Method for Obtaining Nanoparticles

The methods of the invention are a variation of the method described by Brust-Schiffrin (Brust, M.; Fink, J.; Bethell, D.; Schiffrin, D.; Kiely, C. J. J. Chem. Soc., Chem. Commun., 1995, 16, 1655-1656).

The two-phase mixture can be prepared by providing, on one hand, a polar solution (normally aqueous solution) of copper salt and on the other a solution of the transfer agent in a non-polar solvent (normally an organic solvent).

In principle it would be possible to use any solvent for the polar solution which is capable of dissolving the copper salt and is at the same time immiscible with the non-polar phase. Water or a mixture of water with other polar solvents is normally used as the polar solvent. According to a particular embodiment, said copper salt is Cu(NO₃)₂, Cu(NO₃)₂.3H₂O, CuCl₂, CuCO₃, or Cu (C₂O₂H₃)₂.H₂O. According to a particular embodiment, said copper salt is copper (II) salt, for example, hydrated or anhydrous copper nitrate (Cu(NO₃)₂.3H₂O or Cu(NO₃)₂). In the case of the method for obtaining superparamagnetic nanoparticles, the concentration of the copper salt can vary between 10⁻⁶ and 1 M, for example between 10⁻⁴ M and 10⁻¹ M. According to a particular embodiment, the concentration of copper salt is between 5·10⁻⁴ M and 5·10⁻³ M. In the case of the method for obtaining ferromagnetic nanoparticles, the concentration of the copper salt must be greater than 10⁻³ M. According to a particular embodiment, the concentration of copper salt is between 10⁻³ and 10⁻¹ M and, preferably between 10⁻³ and 10⁻² M.

Organic solvents selected from the aromatic solvents (for example, benzene, toluene or xylenes) are normally used for the non-polar solution, although other non-polar organic solvents would also be suitable. The transfer agent allows transporting the ions from the copper to the organic phase so that they can then be trapped by the surfactant. The person skilled in the art knows said transfer agents which can be found in reference books such as, for example, Jean-Louis Salager, “Surfactants. Types and Uses”, Universidad de los Andes, Mérida (Venezuela) 2002, which is entirely incorporated by reference. According to a particular embodiment, the transfer agents used in the present invention are tetraalkylamine salts, for example salts of formula A⁻R₄N⁺, where R has the meaning mentioned above, and A is an anion, preferably fluoride, chloride, bromide or iodide. Each R group is individually selected preferably from the group consisting of alkyls with 6 to 15 carbon atoms. According to a particular embodiment, the transfer agent used is tetraoctylammonium bromide (TOAB), cetyl trimethyl ammonium bromide, [(C₁₆H₃₃)N(CH₃)₃Br]), or tetrabutylammonium bromide. According to another particular embodiment, said transfer agent is a phosphonium halide, oleic acid, or octadecylamine. The concentration of the transfer agent in the non-polar solution tends to be comprised between 10⁻⁶ and 1 M, for example between 10⁻⁴M and 10⁻² M.

In a particular embodiment, the concentration of copper salt is between 10⁻⁴ M and 10⁻² M.

The ratio of both the solutions (or phases) in the two-phase mixture tends to vary between 1:6 and 6:1 polar:non-polar ratio. According to a particular embodiment, the ratio of polar:non-polar phases is between 1:1 and 1:5, preferably between 1:2 and 1:3. Once the two-phase mixture is obtained the transfer of the copper ions to the organic phase which is preferably achieved under stirring and which tends to take between seconds and hours must be waited out. Typical transfer times are between 5 and 120 minutes, normally between 10 and 60 minutes. In any case it can be observed that the transfer has been completed by means of common techniques, the simplest being tracking the loss of blue color of the aqueous phase due to the loss of Cu(II) ions. Logically other techniques such as ICP can be also used.

Once the transfer is completed the surfactant is added in the presence of a reducing agent. The amount of surfactant is greater than that of copper and it is typically added in a surfactant:copper by weight ratio which is greater than 5:1, preferably greater than 8:1, preferably between 8:1 and 15:1. The reducing agents useful for reducing the copper cations to copper metal are known and the person skilled in the art can look them up in reference documents such as, for example, David R. Lide, CRC Handbook of Chemistry and Physics: A Ready-reference Book of Chemical and Physical Data, CRC Press, 1995. In a particular embodiment, hydrides are used as reducing agents. According to a particular embodiment, the hydride is a borohydride, for example, sodium borohydride (NaBH₄). The amount of reducing agent is not particularly relevant and must be sufficient as to reduce all the copper to copper metal.

Once the copper is reduced (approximately between one and ten hours) a two-phase mixture is obtained where the nanoparticles of the invention are in the non-polar phase, which are isolated according to common methods (see for example, Brust, M.; Fink, J.; Bethell, D.; Schiffrin, D.; Kiely, C. J. J. Chem. Soc., Chem. Commun., 1995, 16, 1655-1656). Also see B L Cushing, V L Kolesnichenko, C J O'Connor, “Recent Advances in the Liquid-Phase Syntheses of Inorganic Materials”, Chem. Rev. 104, 3893-3946 (2004); or Xun Wang, Jing Zhuang Qing Peng & Yadong Li, “A general strategy for nanocrystal synthesis”, Nature 437, 121-124 (2005) for other isolation methods.

### Uses of the Nanoparticles of the Invention

### Pharmaceutical Compositions and Kits

The present invention provides pharmaceutical compositions or kits comprising the nanoparticles of the invention.

Examples of pharmaceutical compositions include any solid composition (tablets, pills, capsules, pellets, etc.) or liquid composition (solutions, suspensions or emulsions) for oral, topical or parenteral administration (sterile solutions, suspensions or lyophilized products in a suitable unit dosage form). They can contain active ingredients or other materials with biomedical applications; conventional excipients known in the art, such as binding agents, for example syrup, acacia, gelatin, sorbitol, tragacanth, or polyvinylpyrrolidone; fillers, for example lactose, sugar, corn starch, calcium phosphate, sorbitol or glycine; lubricants for preparing tablets, for example magnesium stearate; disintegrating agents, for example starch, polyvinylpyrrolidone, sodium glycolate of starch or microcrystalline cellulose; or pharmaceutically acceptable wetting agents such as sodium lauryl sulfate.

The formulations mentioned will be prepared using common methods such as those described or referred to in the Spanish and United States Pharmacopeias and in similar reference texts.

The nanoparticles of the invention can be applied by means of any suitable method, such as intravenous infusion, oral preparations and intraperitoneal and intravenous administration. They can be used with other drugs for providing a combined therapy. The other drugs can form part of the same composition or can be provided as a separated composition for administration at the same time or at different times.

In the present invention, a “kit” is understood as a product comprising the nanoparticles of the invention and additional therapeutic agents forming the composition packed such that it allows the transport, storage and the simultaneous or successive administration thereof. Therefore the kits of the invention can contain one or more suspensions, tablets, capsules, inhalers, syringes, patches and the like which contain the nanoparticles of the invention and which can be prepared in a single dose or as multiple doses. The kit can additionally contain a suitable carrier for resuspending the compositions of the invention such as aqueous media such as saline solution, Ringer's solution, lactated Ringer's solution, dextrose and sodium chloride, water-soluble media such as alcohol, polyethylene glycol, propylethylene glycol and non water-soluble carriers such as corn oil, cottonseed oil, peanut oil, sesame oil, ethyl oleate, isopropyl myristate and benzyl benzoate. Another component which can be present in the kit is a container which allows maintaining the formulations of the invention within the determined limits. Materials suitable for preparing such containers include glass, plastic (polyethylene, polypropylene, polycarbonate and the like), bottles, vials, paper, envelopes and the like.

The kit of the invention can additionally contain instructions for the simultaneous, successive or separated administration of the different pharmaceutical formulations present in the kit. Said instructions can be in the form of printed material or in the form of electronic support which can store the instructions such that they can be read by a subject, such as electronic storage means (magnetic discs, tapes and the like), optical means (CD-ROM, DVD) and the like. The means can additionally or alternatively contain internet web pages providing said instructions.

### Therapeutic Uses

The smaller dimensions of the nanoparticles of the invention means that they have unique physical properties. Their small size turns them into systems ideal for use in biological applications (for example, see US 2008/0089836 or WO 2005/09704).

The capacity of the nanoparticles of the invention to generate an energy transfer allows application in the treatment of tumors by means of hyperthermia (see for example A Topical Review of Magnetic Fluid Hyperthermia. Jennifer L. Phillips). Magnetic hyperthermia is one of the few methods having the potential theoretical possibility of causing very localized damage in the tumor without damaging the adjacent healthy tissue. The magnetic nanoparticles convert the electromagnetic energy into heat when they are exposed to external radiofrequency (RF) fields such that the ferromagnetic and superparamagnetic nanoparticles of the invention can be applied for obtaining a controlled heat in carcinogenic tumors, opening up new possibilities in cancer therapy.

Like the systems designed for massive state ferromagnetic materials (see e.g. A. Jordan et al., J. Mag. Mag. Mat. 225 (2001) 118-126; or U.S. Pat. No. 6,001,054), these systems consist of an AC magnetic field generator perpendicular to the axial direction of the patient. The system additionally comprises an adjustable AC frequency in the range of 100 kHz and a variable field intensity of 0 to 15 kA/m.

There are two different strategies for the purpose of putting the nanoparticles of the invention in the desired area. A first option is to incorporate ligands which specifically recognize a target cell on the surface of the nanoparticles. In a second strategy the nanoparticles are sent to the area of interest by means of an external magnetic field.

They also have application as contrast agents in systems for obtaining images by means of magnetic resonance (Magnetic Resonance Imaging), or the analysis of biological samples by means of optical and electron spectroscopy, improving on the viewing efficiency (see for example Lee Josephson, “Magnetic Nanoparticles for MR Imaging” BioMEMS and Biomedical Nanotechnology, Ed. Springer, US (2007)). The improvement in resolution which the nanoparticles of the invention would provide can allow detecting small tumors and therefore better treatment possibilities.

The nanoparticles of the invention also have application in the transport and/or immobilization, as well as in the controlled release of active ingredients in a biological medium (see for example Stuart C McBain, Humphrey H P Yiu, and Jon Dobson, “Magnetic nanoparticles for gene and drug delivery” Int J Nanomedicine. 3(2), 169-180 (2008)). The nanoparticles of the invention can be used as the tracers of drug release instead of radioactive materials used, which allow monitoring the release of a drug through the measurement of magnetic property variations, eliminating the harmful effects of radiation. Additionally, they can be used in vaccination guns as an alternative to the vaccine injectors which are commonly compressed air or gas (particularly helium), causing pain and leaving marks on the skin. The injection power would in this case be provided by applying a magnetic field, which would cause the nanoparticles to speed up in their passage through the epidermis.

The nanoparticles of the invention can also be used for preparing biosensors and biochips (see for example Tarl W. Prow, Jose H. Salazar, William A. Rose, Jacob N. Smith, Lisa Reece, Andrea A. Fontenot, and Nan A. Wang, R. Stephen Lloyd, James F. Leary “Nanomedicine: nanoparticles, molecular biosensors, and targeted gene/drug delivery for combined single-cell diagnostics and therapeutics” Proc. SPIE, Vol. 5318, 1 (2004)). In these systems the nanoparticles can act as markers signaling the presence of specific elements. Nanoparticles of the invention comprising ligands capable of recognizing a specific biomolecule can, for example, be prepared. When said biomolecule is present the nanoparticles are fixed such that their signal is captured by a magnetic sensor which is separated from the nanoparticles by a protective passivation layer.

The nanoparticles of the invention can also be used for separating previously labelled cells by following the methods described in paragraph [0334] of US 2008/0089836.

Other documents which mention possible applications of nanoparticles include “Biomedical Applications of Nanotechnology”, Ed. By V. Labhasetwar and D. L. Leslie-Pelecky, Wiley-Interscience, 2007; “Biological and Biomedical Nanotechnology”, A. P. Lee and L. J. Lee Editors, Springer US (2007); or Challa S. S. R. Kumar, “Nanomaterials for cancer therapy” Ed. Wiley-VCH (2006)

### Non-Therapeutic Uses

The nanoparticles of the invention are also useful as catalysts for synthesizing carbon nanotubes and inorganic nanowires, or for preparing ultrathin systems for magnetic data storage. Details about these uses or other similar uses can also be found in US 2008/0089836, U.S. Pat. No. 6,521,773 or U.S. Pat. No. 6,534,039.

An ordered distribution of nanoparticles of the invention on a support serves as a base for fabricating compact discs which use magnetic fields for storing data (see for example, Günter Reiss & Andreas Hütten, “Magnetic nanoparticles: Applications beyond data storage”, Nature Materials 4, 725-726 (2005)). The information can in turn be read with a magnetic sensor (magnetoresistive type) by Kerr effect using a laser.

### EXAMPLES

Specific embodiments of the invention which in no case must be considered limiting are presented below.

### Example 1

### Synthesis of Superparamagnetic Nanoparticles

30 ml of a 3×10⁻³ M aqueous Cu(NO₃)₂ solution were mixed with 80 ml of a 5×10⁻³ M TOAB (tetraoctylammonium bromide) solution in toluene to obtain the nanoparticles of the invention. This two-phase mixture was kept under stirring until the transfer of the Cu(II) ions to the organic phase (loss of the blue color in the aqueous phase after about 30 minutes) was observed, then 249 μl of 1-dodecylamine (C₁₂H₂₇N) and subsequently 25 ml of a recently prepared 0.4 M aqueous NaBH₄ solution were added. Said solution was kept under stirring for approximately 3 hours, a two-phase mixture formed by a dark colloidal toluene solution formed by 1-dodecylamine-surrounded copper nanoparticles (Cu—NHR) and a completely colorless aqueous solution being obtained. After separating both phases the aqueous phase was discarded. The volume of the toluene solution was reduced to 10 ml in a rotary evaporator for subsequently adding 400 ml of ethanol for the purpose of destabilizing the solution. The mixture was put in a freezer and maintained at −6° C. for 18 h to precipitate the nanoparticles of the invention. Said nanoparticles were finally filtered under vacuum with a number 5 sieve plate, about 8 mg of a waxy dark colored product having superparamagnetic character at room temperature being obtained. The particles obtained had an average size of 10 nm and a homogeneous particle distribution.

### Example 2

### Synthesis of Ferromagnetic Nanoparticles

30 ml of a 3×10⁻³ M aqueous Cu(NO₃)₂ solution is mixed with 80 ml of a 5×10⁻³ M TOAB (tetraoctylammonium bromide) solution in toluene. This two-phase mixture is kept under stirring until the transfer of the Cu(II) ions to the organic phase (about 30′) is observed, then 250 μl of 1-dodecanethiol (C₁₂H₂₆S) and subsequently 25 ml of a recently prepared 0.4 M aqueous NaBH₄ solution are added. Said solution is kept under stirring for approximately 3 hours, a two-phase mixture formed by a dark colloidal toluene solution formed by dodecanethiol-surrounded copper nanoparticles (Cu—SR) and a completely colorless aqueous solution being obtained. After separating both phases the aqueous phase is discarded. The volume of the toluene solution is reduced to 10 ml in a rotary evaporator for subsequently adding 400 ml of ethanol for the purpose of destabilizing the solution. The mixture is put into a freezer and maintained at −6° C. for 18 h, an interval which assures selective Cu nanoparticle precipitation. Said nanoparticles are finally filtered under vacuum with a number 5 sieve plate, about 15 mg of a waxy dark colored product having ferromagnetic character at room temperature being obtained. The thermogravimetric analysis and the elemental analysis (C, H and S) thereof shows an organic matter content of 32.17% with a C, H and S composition of 23.05%, 3.92% and 5.2%, respectively.

### Example 3

### Measuring and Calculating the Average Size of the Nanoparticles of the Invention

A transmission electronic microscopy (TEM) study was conducted for the purpose of knowing the size of the particles, their size distribution and their morphology. The size of the nanoparticles was obtained from the micrographs taken in the Philips CM200 transmission electron microscope equipped with EDX and WDX microanalysis set up in the General Electronic Microscopy and Microanalysis Service of the Faculty of Science and Technology (UPV/EHU). It was operated at 200 kV using a simple inclination sample holder both for the image and for the diffraction.

To analyze the samples, several drops of the different colloidal mixtures formed by the nanoparticles and the toluene solvent were taken and deposited on several Cu strainers with carbon coating on which the dispersed particles are adhered. Several hours are waited out so that the solvent which may remain in the strainer is evaporated and it was observed under the microscope.

The particles of each of the TEM images were analyzed with the aid of the ImageJ program, [W. Rasband (National Institutes of Health, NIH), (http://rsb.info.nih.gov/ij/)] image processing program to estimate the particle size and its distribution. A log-normal (or log-Gaussian) distribution or a Gaussian distribution was used for adjusting the size distributions.

The normal or Gaussian function is defined as:

$$f ( r ) = \frac{1}{\sqrt{2 π} \cdot r σ} exp ( - \frac{{( r - {r}_{0} )}^{2}}{2 {σ}^{2}} ) \qquad ( II .1 )$$

for r>0 and where r₀ and σ are the mean and the standard deviation of the variable logarithm, respectively.

The form of the log-normal distribution is given by the following mathematical expression:

$$f ( r ) = \frac{1}{\sqrt{2 π} \cdot r σ} exp ( - \frac{{( ln r - {r}_{0} )}^{2}}{2 {σ}^{2}} ) \qquad ( II .2 )$$

for r>0 and where r₀ and σ are the mean and the standard deviation of the variable logarithm respectively, and where the geometric mean is defined as r_{0geom}=exp(r₀).

The results obtained indicate that the nanoparticles obtained had an average size between 2 and 5 nm.

### Example 4

### Measuring the Magnetic Properties of the Nanoparticles of the Invention

To characterize the magnetic properties of the nanoparticles of the invention the magnetism thereof (magnetization vs magnetic field applied) was measured in a Quantum Design MPMS7 SQUID magnetometer (Superconducting quantum interference device) supplying the magnetic field by means of a superconductor coil (7 Tesla maximum field) or in a VSM magnetometer (Vibrating sample magnetometer) between 5 and 300 K.

The ratio existing between the measurement and the actual magnetic moment of the sample was previously determined by calibrating with a standard with a known magnetic moment value, in this case the Pd.

To take the measurements, about 2 mg of solid-state sample which was introduced in a capsule made of a polymer with a small amount of cotton were weighed. The capsule was placed in a tube made of the same polymer which was placed in a metal rod and was introduced in the SQUID.

### Example 5

### Calculating of Organic Matter Content

Dynamic thermogravimetric measurements, i.e., heating the sample by following a predetermined program, normally linear, at a variable temperature were performed to calculate the amount of organic matter which the nanoparticles of the invention lost.

Therefore, 2-3 mg of the mass of the nanoparticles of the invention deposited in an aluminum boat and placed in a TA Instruments SDT2960 Simultaneous DSC/TGA thermobalance which had previously been tare weighed were used. The sample was subjected to a heating rate of 10°/min until 800° C. in an Ar atmosphere.

1. A method for preparing a ferromagnetic nanoparticle which comprises
(i) providing a two-phase mixture of water and an organic solvent comprising a copper salt and a transfer agent;
(ii) adding a surfactant of formula RSH, wherein R is a hydrophobic group in the presence of a reducing agent; and
(iii) precipitating the nanoparticles formed
said method being characterized in that the concentration of the copper salt in the aqueous phase is greater than 10⁻³ molar.

2. A method for preparing a superparamagnetic nanoparticle which comprises
(i) providing a two-phase mixture of water and an organic solvent comprising a copper salt and a transfer agent;
(ii) adding a surfactant of formula RNH₂, wherein R is a hydrophobic group in the presence of a reducing agent; and
(iii) precipitating the nanoparticles formed.

3. The method according to 1, wherein the concentration of the copper salt is between 10⁻³ and 10⁻¹ and preferably between 10⁻³ and 10⁻² M.

4. The method according to claim 1 wherein the copper salt is selected from the group consisting of Cu(NO₃)₂, Cu(NO₃)₂.3H₂O, CuCl₂, CuCO₃, and Cu(C₂O₂H₃)₂.H₂O, preferably Cu(NO₃)₂.3H₂O and Cu(NO₃)₂.

5. The method according to claim 1 wherein the transfer agent is selected from the group consisting of a phosphonium halide, a quaternary ammonium, oleic acid, and octadecylamine.

6. A ferromagnetic nanoparticle obtainable by the method defined in claim 1.

7. A superparamagnetic nanoparticle with an average diameter less than 30 nm, characterized in that it comprises copper associated with a surfactant of formula RNH₂, wherein R is a hydrophobic group.

8. The nanoparticle according to claim 7, wherein R is a hydrocarbon chain between 6 and 30 carbon atoms.

9. The nanoparticle according to claim 8, wherein R is selected from the group consisting of alkyl, alkenyl, and alkynyl with 6 to 30 carbon atoms.

10. The nanoparticle according to claim 6, with an average diameter comprised between 2 and 5 nm.

11. The nanoparticle according to claim 6 for use as a medicament.

12. The nanoparticle according to claim 6 for preparing a medicament or kit for the transportation and/or immobilization of active ingredients in a biological medium, the controlled release of active ingredients, the treatment of tumors by means of hyperthermia, contrast agents in systems for obtaining images by means of magnetic resonance, or the analysis of biological samples by means of optical and electron spectroscopy.

13. Use of a nanoparticle according to claim 6 as a catalyst for synthesizing carbon nanotubes and inorganic nanowires, preparing biosensors and biochips, or for preparing ultrathin systems for magnetic data storage.

14. The method according to claim 2, wherein the concentration of the copper salt is between 10⁻³ and 10⁻¹ and preferably between 10⁻³ and 10⁻² M.

15. The method according to claim 2, wherein the copper salt is selected from the group consisting of Cu(NO₃)₂, Cu(NO₃)₂.3H₂O, CuCl₂, CuCO₃, and Cu(C₂O₂H₃)₂.H₂O, preferably Cu(NO₃)₂.3H₂O and Cu(NO₃)₂.

16. The method according to claim 2, wherein the transfer agent is selected from the group consisting of a phosphonium halide, a quaternary ammonium, oleic acid, and octadecylamine.

17. The nanoparticle according to claim 7, with an average diameter comprised between 2 and 5 nm.

18. The nanoparticle according to claim 7 for use as a medicament.

19. The nanoparticle according to claim 7 for preparing a medicament or kit for the transportation and/or immobilization of active ingredients in a biological medium, the controlled release of active ingredients, the treatment of tumors by means of hyperthermia, contrast agents in systems for obtaining images by means of magnetic resonance, or the analysis of biological samples by means of optical and electron spectroscopy.

20. Use of a nanoparticle according to claim 7 as a catalyst for synthesizing carbon nanotubes and inorganic nanowires, preparing biosensors and biochips, or for preparing ultrathin systems for magnetic data storage.
